(19) United States
(12) Patent Application Publication
(10) Pub. No.: US 2019/0099089 A1
(43) Pub. Date: Apr. 4, 2019

(54) IMAGE ANALYSIS APPARATUS AND IMAGE ANALYSIS METHOD

(71) Applicant: SONY CORPORATION, TOKYO (JP)

(72) Inventors: TETSURO KUWAYAMA, TOKYO (JP); HIROSHI ICHIKI, KANAGAWA (JP)

(21) Appl. No.: 16/085,850

(22) PCT Filed: Jan. 11, 2017
(86) PCT No.: PCT/JP2017/000570

§ 371 Date: Sep. 17, 2018

(30) Foreign Application Priority Data
Mar. 25, 2016 (JP) 2016-062751

(51) Int. Cl. A61B 5/026; A61B 5/02; G02B 27/48; G01N 21/47

(52) U.S. Cl. CPC A61B 5/0261; A61B 5/02007; G01N 2021/479; G01N 21/4788; G02B 27/48

(57) ABSTRACT

Provided is an image analysis technique capable of analyzing a fluid irrespective of the motion of an imaging object including a fluid and showing motion such as pulsations and beats even when the imaging object is analyzed by using speckle. An image analysis apparatus includes: a light source that irradiates an imaging object with laser light having a controlled wavelength; a modulation unit that modulates intensity of the laser light emitted from the light source; a speckle imaging unit that captures a speckle image obtained from scattered light of the imaging object irradiated with the laser light; a synchronization unit that synchronizes irradiation by the light source and imaging by the speckle imaging unit; and an analysis unit that analyzes the speckle image captured by the speckle imaging unit.

### TECHNICAL FIELD

The present technology relates to an image analysis apparatus. More particularly, the present technology relates to an image analysis apparatus and an image analysis method that use speckle generated by irradiating an imaging object with light.

### BACKGROUND ART

In the past, in order to grasp the shape, structure, and the like of a biological sample such as a blood vessel or a cell, an image analysis apparatus and an image analysis method using an optical method have been developed.

Additionally, in an imaging technique using an optical method in the case of using a flow path such as a blood vessel as an imaging object, there is a concern that occurrence of various noises may cause detection accuracy to deteriorate. As one of the noises, speckle is known. The speckle is a phenomenon that a spot-like pattern appears on an irradiated surface depending on an uneven shape of the irradiated surface. In recent years, techniques have also been developed with respect to a method of imaging a flow path such as a blood vessel by using speckle which is one of the noises.

By the way, speckle is a random interference/diffraction pattern due to scattering or the like in an optical path. In addition, the magnitude of speckle is represented by an index called speckle contrast which is a value obtained by dividing the standard deviation of the intensity distribution by the average of the intensity distribution. When the imaging object irradiated with coherent light is observed by using an imaging optical system, the speckle caused by scattering of the imaging object is observed on the image plane. When the imaging object moves or changes in shape, a random speckle pattern corresponding to the movement or change is observed.

When a light scattering fluid such as blood is observed, the speckle pattern changes at every moment according to the change in fine shape caused by the flow. At that time, when an imaging element is arranged on the image plane and the fluid is imaged in an exposure time sufficiently longer than the change of the speckle pattern, the speckle contrast of a portion in which the blood is flowing, that is, a portion of the blood vessel is reduced in time average. Angiography can be performed by using such a change in speckle contrast.

As an image analysis technique using the speckle as described above, a rheometer disclosed in Patent Literature 1 is known (see Patent Literature 1).

This rheometer includes an irradiation system for irradiating blood corpuscles of a biological tissue with laser light, and a solid-state imaging element for optically storing image information based on reflected light from the biological tissue and continuously reading the optically stored image information at predetermined time intervals, the rheometer sequentially storing the pieces of image information of a plurality of frames read from the solid-state imaging element and calculating a blood flow state of the blood corpuscles on the basis of each stored image signal, the rheometer being characterized by being configured to intermittently perform irradiation with laser light at time intervals shorter than the predetermined time intervals.

Furthermore, as another image analysis technique using speckle, a blood flow image diagnosing device disclosed in Patent Literature 2 is known (see Patent Literature 2).

This blood flow image diagnosing device is a device obtained by adding a function of analyzing an obtained blood flow map to a blood flow speed visualizing device including: a laser beam irradiating system for irradiating an observation region of a biological tissue having blood corpuscles with a laser beam; a light-receiving system including a light-receiving unit adapted to detect light reflected from the observation region of the biological tissue and including a large number of pixels; an image capturing unit for continuously capturing multiple images for a predetermined time equal to or longer than one heart beat on the basis of the signals from the light-receiving unit; an image storing unit for storing the multiple images; a computing unit for computing the blood flow speed in the biological tissue from the temporal variation of the output signals of the corresponding pixels of the stored images; and a display unit for displaying the two-dimensional distribution of the computation results as a blood flow map, the blood flow image diagnosing device being characterized in that the computing unit has a function of separating the blood flows within the blood vessels appearing on the superficial portion of the observation region of the biological tissue (superficial blood vessels) and the blood flows at the background therearound (background blood flows) from pieces of blood flow map data about one or more heart beats, the display unit has a function of displaying the blood flows at the respective portions separately on a blood flow map, the computing unit has a function of computing and comparing information regarding the blood flow, such as a blood flow value, a blood flow waveform, and a blood vessel diameter of each portion, and the display unit is provided with a function of displaying those computing results.

### CITATION LIST

### Patent Literature

Patent Literature 1: Japanese Patent Application Laid-open No. Hei 08-112262

Patent Literature 2: WO 2010/131550

### DISCLOSURE OF INVENTION

### Technical Problem

However, the image analysis technique described in Patent Literature 1 has a problem that if the exposure time is actually shortened enough to suppress fluctuation of the speckle signals, the exposure amount becomes significantly small, which is inadequate to realistic imaging conditions. In addition, there is another problem that it is very difficult to perform imaging with a high-resolution imaging element because frame intervals are made very short. Moreover, there is another problem that, in a case where the intensity of the output of the laser light source is modulated within a short time, a laser oscillation wavelength sways due to an internal temperature change, which reduces contrast of a speckle pattern on the entire screen.

Further, in the image analysis technique described in Patent Literature 2, for example, assuming that a pulsating/beating biological tissue and a blood flow of a blood vessel of the biological tissue are an imaging object, fluid imaging using speckle catches sway of the entire screen, the motion of the biological tissue, and the like while being a method having very high sensitivity to the motion of the biological tissue, with the result that the blood flow itself is hard to catch.

In this regard, it is a main object of the present technology to provide an image analysis technique capable of analyzing a fluid irrespective of the motion of an imaging object including a fluid and showing motion such as pulsations and beats even when the imaging object is analyzed by using speckle.

### Solution to Problem

The present technology provides an image analysis apparatus including: a light source that irradiates an imaging object with laser light having a controlled wavelength; a modulation unit that modulates intensity of the laser light emitted from the light source; a speckle imaging unit that captures a speckle image obtained from scattered light of the imaging object irradiated with the laser light; a synchronization unit that synchronizes irradiation by the light source and imaging by the speckle imaging unit; and an analysis unit that analyzes the speckle image captured by the speckle imaging unit.

The image analysis apparatus may further include an exposure control unit that controls an exposure time for the imaging object.

Further, in the image analysis apparatus, the light source may be configured to irradiate the imaging object with the laser light within the exposure time for the imaging object.

Furthermore, in the image analysis apparatus, the exposure control unit may employ a global shutter system. Alternatively, the exposure control unit may employ a rolling shutter system.

Further, in the image analysis apparatus, the exposure time for the imaging object may be 32.2 ms or less.

Furthermore, in the image analysis apparatus, the light source may be a distributed feedback semiconductor laser light source or a grating feedback semiconductor laser light source.

Further, the present technology also provides an image analysis method including: a light irradiating step of irradiating an imaging object with laser light having a controlled wavelength; a modulating step of modulating intensity of the laser light; a synchronizing step of synchronizing irradiation with the laser light and imaging by a speckle imaging unit; a speckle imaging step of capturing a speckle image obtained from scattered light of the imaging object irradiated with the laser light; and an analyzing step of analyzing the speckle image captured by the speckle imaging unit.

### Advantageous Effects of Invention

According to the present technology, it is possible to analyze a fluid irrespective of the motion of an imaging object including a fluid and showing motion such as pulsations and beats even when the imaging object is analyzed by using speckle, and thus improve accuracy to analyze the state of the imaging object.

Note that the effects described herein are not necessarily limited and may be any of the effects that are intended to be described in the present technology.

### BRIEF DESCRIPTION OF DRAWINGS

FIG. 1 is a schematic conceptual diagram schematically showing a concept of a first embodiment of an image analysis apparatus according to the present technology.

FIG. 2 is a block diagram showing details of the image analysis apparatus shown in FIG. 1.

FIG. 3 is a schematic diagram showing a first modified example of the image analysis apparatus of the first embodiment shown in FIG. 1.

FIG. 4 is a schematic diagram showing a second modified example of the image analysis apparatus of the first embodiment shown in FIG. 1.

FIG. 5 is a timing chart of the image analysis apparatus shown in FIG. 1.

FIG. 6 is a graph substituting diagram showing a relationship between speckle contrast, an object speed, and an exposure time for an imaging object.

FIG. 7 is a schematic conceptual diagram schematically showing a concept of a second embodiment of the image analysis apparatus according to the present technology.

FIG. 8 is a schematic diagram showing a first modified example of the image analysis apparatus of the second embodiment shown in FIG. 7.

FIG. 9 is a schematic diagram showing a second modified example of the image analysis apparatus of the second embodiment shown in FIG. 7.

FIG. 10 is a first example of a timing chart of the image analysis apparatus shown in FIG. 7.

FIG. 11 is a second example of a timing chart of the image analysis apparatus shown in FIG. 7.

FIG. 12 is a schematic conceptual diagram schematically showing a concept of a third embodiment of the image analysis apparatus according to the present technology.

FIG. 13 is a timing chart of the image analysis apparatus shown in FIG. 12.

FIG. 14 is a flowchart of an image analysis method according to the first embodiment of the present technology.

FIG. 15 is a flowchart of an image analysis method according to the second embodiment of the present technology.

### MODE(S) FOR CARRYING OUT THE INVENTION

Suitable embodiments for implementing the present technology will be described below with reference to the drawings. Each embodiment to be described below shows an example of a representative embodiment of the present technology, so that the scope of the present technology is not to be narrowly interpreted by the embodiments. Note that the description will be made in the following order.

1. Image Analysis Apparatus according to First Embodiment

(1) Light Source

(2) Modulation Unit
(3-1) Exposure control unit

(3) Speckle Imaging Unit

(4) Synchronization Unit
(4-1) Modified Example of Synchronization Unit

(5) Analysis Unit

(6) Storage Unit

(7) Display Unit

(8) Imaging Object

2. Image Analysis Apparatus according to Second Embodiment

(1) Modified Example of Image Analysis Apparatus according to Second Embodiment

3. Image Analysis Apparatus according to Third Embodiment

(1) Exposure-time Change Unit

4. Image Analysis Method according to First Embodiment

(1) Modulating Step

(2) Synchronizing Step

(3) Light Irradiating Step

(4) Speckle Imaging Step

(5) Analyzing Step

(6) Storing Step

(7) Displaying Step

5. Image Analysis Method according to Second Embodiment

(1) Exposure-time Changing Step

### 1. Image Analysis Apparatus according to First Embodiment

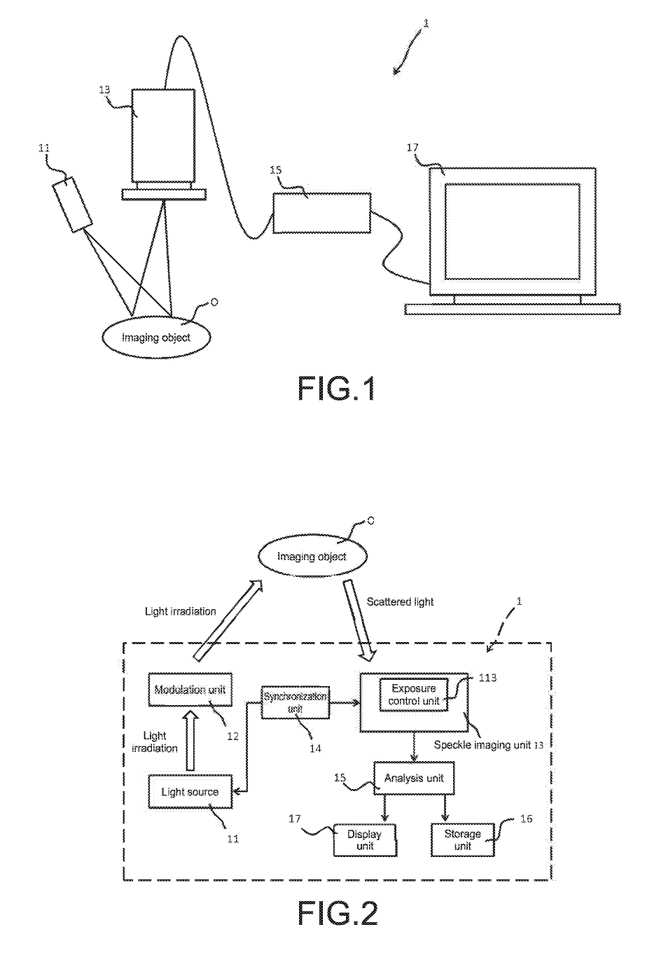

A first embodiment of an image analysis apparatus according to the present technology will be described with reference to FIGS. 1 to 6.

An image analysis apparatus 1 shown in FIGS. 1 and 2 includes at least a light source 11, a modulation unit 12, a speckle imaging unit 13, a synchronization unit 14, and an analysis unit 15. In addition, as necessary, the image analysis apparatus 1 may further include a storage unit 16, a display unit 17, and the like. Each component will be described in detail below.

### (1) Light Source

The light source 11 irradiates an imaging object O with coherent light.

The coherent light emitted from the light source 11 denotes light in which the phase relationship between light waves at arbitrary two points in a light flux is invariable and constant in terms of time and, thus, even in the case of dividing the light flux by an arbitrary method and, after that, providing a large optical path difference and overlaying the divided light fluxes again, perfect coherency is exhibited.

Laser light is favorable as the coherent light. As the light source 11 that emits laser light, for example, an argon ion (Ar) laser, a helium-neon (He—Ne) laser, a dye laser, a krypton (Cr) laser, a distributed feedback (DFB) or grating feedback semiconductor laser, and the like may be used. Of those, it is favorable to use a semiconductor laser in which a wavelength to be output is controlled.

Further, a modulation frequency of the light intensity output from the light source 11 is not particularly limited, but the modulation frequency needs to be adequate for speckle imaging. For example, in order to present speckle as a moving image, a modulation frequency of 24 Hz or more is favorable. In order that a user perceives a sufficiently smooth moving image, a modulation frequency of 120 Hz or more is more favorable.

Furthermore, a time during which the imaging object O is exposed to light by light irradiation of the light source 11 only needs to be set such that pulsations/beats of the imaging object shown in the background of a fluid can be suppressed and an analysis of the fluid can be performed, for example.

Here, the speckle contrast is known to change depending on the presence/absence of the motion of the imaging object O and to increase in a state where the imaging object O is at rest and decrease in a state where the imaging object O is moving. For this reason, as shown in FIG. 6, in a case where the speed of the imaging object O is high and a numerical value of the exposure time (e.g., 66.6 ms) is large, a numerical value of the speckle contrast also decreases.

For example, in a case where the imaging object O is a biological sample such as a pulsing/beating heart and a state of a blood vessel of the biological sample is analyzed using speckle, it is generally understood that an arteriole has a blood flow speed of approximately 50 mm/s, whereas the pulsation/beat has approximately 1 to 5 mm/s.

Therefore, in the image analysis apparatus according to the present technology, the exposure time for the imaging object O is favorably set to 32 ms or less, more favorably, 16.6 ms or less, and further favorably, 3.33 ms or less (see FIG. 6).

### (2) Modulation Unit

The image analysis apparatus 1 according to the present technology includes the modulation unit 12 that modulates the intensity of the laser light emitted from the light source 11.

A method of modulating the intensity by the modulation unit 12 is not particularly limited. For example, a method of changing the magnitude of a current supplied to the light source by using a semiconductor laser as the light source 11 (direct intensity modulating method), a method of externally adding modulation to laser light that is output from a semiconductor laser being the light source 11 (external modulation method), and the like are exemplified.

Note that the image analysis apparatuses shown in FIGS. 2 to 4 each have a configuration employing the external modulation method, in which the modulation unit 12 is provided separately from the light source 11.

In other words, for the configuration of the modulation unit 12 according to the present technology, a configuration incorporated in the light source 11 and a configuration provided outside the light source 11 are conceivable.

For the configuration in which the modulation unit 12 is incorporated in the light source 11, for example, the above-mentioned distributed feedback (DFB) or grating feedback semiconductor laser is exemplified.

Meanwhile, for the configuration in which the laser light output from the light source 11 is modulated, for example, a MEMS (Micro Electro Mechanical Systems), an AOM (Acousto-Optic Modulator), an EOM (Electro-Optic Modulator), a liquid-crystal shutter, a mechanical shutter, a chopper, and the like are exemplified.

### (3) Speckle Imaging Unit

In the speckle imaging unit 13, imaging of speckle appearing on a surface of the imaging object O is performed on the basis of scattered light obtained from the imaging object O irradiated with the laser light of each light source 11.

This speckle imaging unit 13 includes an imaging optical system that forms an image of the scattered light obtained from the imaging object O, and an imaging system that receives the light of the image formed by the imaging optical system. The imaging optical system includes an imaging element such as a CCD sensor or a CMOS sensor, an imaging lens, and the like. In the CMOS sensor, a global shutter system and a rolling shutter system are known, and any of the systems can be employed in the image analysis apparatus 1 according to the present technology.

### (3-1) Exposure Control Unit

In the image analysis apparatus 1 according to the present technology, the speckle imaging unit 13 includes an exposure control unit 113 that controls an exposure time for the imaging object O. Specifically, the speckle imaging unit 13 has a configuration to adjust a state where all of the pixels in the imaging element are exposed to light when a speckle image is to be captured. More specifically, a CMOS sensor of the global shutter system and a CMOS sensor of the rolling shutter system are exemplified.

In other words, in the present technology, the imaging element of the speckle imaging unit 13 corresponds to the exposure control unit 113.

The imaging method performed by the speckle imaging unit 13 is not particularly limited as long as the effect of the present technology is not impaired, and one or two or more known imaging methods may be selected and used freely in combination. For example, an imaging method using the imaging element described above can be exemplified.

In the speckle imaging unit 13, for example, an image or the like in which a pseudo blood vessel through which pseudo blood flows is mapped on the basis of the speckle is generated. Since the speckle is a random interference/diffraction pattern as described above, when a light scattering fluid such as blood moves or changes with time, the speckle also varies with time. For this reason, it is possible to observe the boundary between the fluid and other portions.

Note that, in order to more clarify the portion where speckle occurs, the speckle imaging unit 13 may have a configuration where, for example, equalization is performed by using a plurality of speckle images to reduce irregularity of the speckle images.

Further, the speckle imaging unit 13 may include a filter that blocks external light so as to be capable of positively taking in the scattered light from the imaging object O.

### (4) Synchronization Unit

The image analysis apparatus 1 according to the present technology includes the synchronization unit 14. In the synchronization unit 14, a laser light irradiation time of the light source 11 and an imaging time of the speckle imaging unit are caused to coincide with each other.

Specifically, the synchronization unit 14 of the image analysis apparatus 1 shown in FIGS. 1 and 2 outputs a synchronization signal for causing the laser light irradiation time of the light source 11 and the imaging time of the speckle imaging unit to coincide with each other to the light source 11 and the speckle imaging unit 13. As a result, in the light source 11 and the speckle imaging unit 13, irradiation with laser light and capturing of a speckle image are simultaneously performed on the basis of the input synchronization signal.

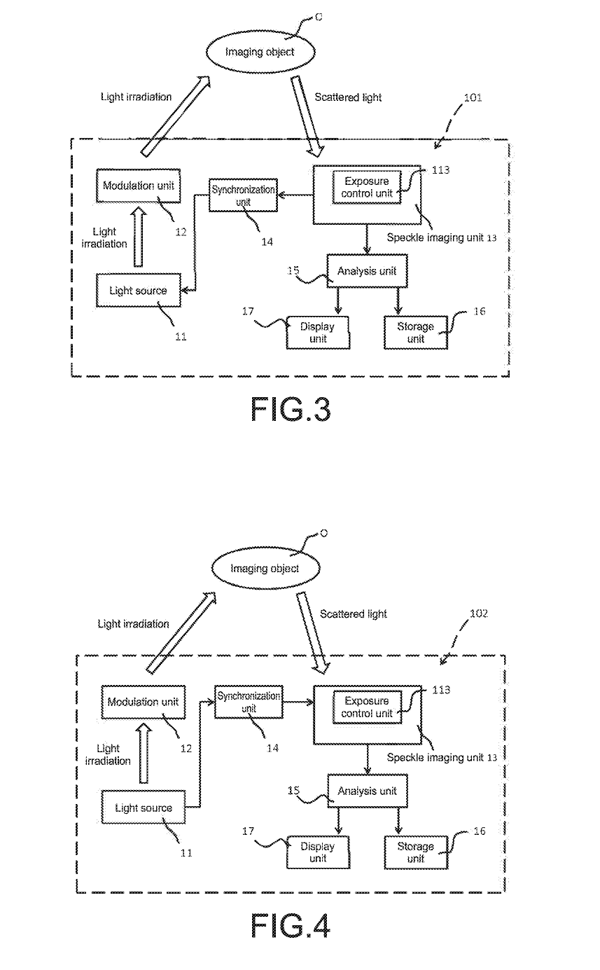

In the present technology, the configuration of the synchronization unit is not limited to the configuration described above, and a known configuration can be employed. Furthermore, the image analysis apparatus 1 shown in FIGS. 1 and 2 has the configuration in which the synchronization signal is output from the synchronization unit 14 to the light source 11 and the speckle imaging unit 13, but the method of acquiring the synchronization signal is not particularly limited. Another example of this method will be described below with reference to FIGS. 3 and 4.

### (4-1) Modified Example of Synchronization Unit

FIG. 3 is a schematic diagram showing a first modified example of the image analysis apparatus of the first embodiment shown in FIG. 1. As is understood from FIG. 3, in this image analysis apparatus 101, the synchronization unit 14 acquires the synchronization signal from the speckle imaging unit 13 and further outputs the synchronization signal to the light source 11.

Meanwhile, FIG. 4 is a schematic diagram showing a second modified example of the image analysis apparatus of the first embodiment shown in FIG. 1. In this image analysis apparatus 102 according to the second modified example, the synchronization unit 14 acquires the synchronization signal from the modulation unit 12 and further outputs the synchronization signal to the speckle imaging unit 13.

In the image analysis apparatuses according to the present technology, the synchronization unit 14 enables the irradiation time of the light source 11 and the imaging time of the speckle imaging unit 13 to coincide with each other, and thus an analysis accuracy using speckle can be enhanced.

### (5) Analysis Unit

The image analysis apparatus 1 according to the first embodiment includes the analysis unit 15 that analyzes a state of the imaging object O on the basis of a speckle image, which is captured by the speckle imaging unit 13.

In this analysis unit 15, for example, an intensity distribution of speckle is measured in a speckle image captured by the speckle imaging unit 13.

Using a result of the measurement, speckle contrast, which is a value obtained by dividing the standard deviation of the intensity distribution by the average of the intensity distribution, is measured. By such measurement of speckle contrast, angiography can be performed by using a change in speckle contrast in a case where the imaging object O is assumed as a blood vessel being a light scattering fluid. Moreover, since the speckle varies with time, the speed of the blood flow can also be analyzed.

Note that, in such a case, the method of measuring the intensity distribution of the speckle or the speckle contrast is not particularly limited as long as the effect of the present technology is not impaired, and one or two or more known measurement methods may be selected and used freely in combination.

### (6) Storage Unit

The imaging apparatus 1 according to the present technology can further include the storage unit 16 that stores the speckle image captured by the speckle imaging unit 13, the speckle contrast measured by the analysis unit 15, the analysis result by the analysis unit 15, and the like as necessary.

This storage unit 16 is not necessarily included in the image analysis apparatus according to the present technology, but the image analysis apparatus may be connected to, for example, an external storage device to store the speckle image and the like.

### (7) Display Unit

The image analysis apparatus according to the present technology can further include the display unit 17 that displays the speckle image captured by the speckle imaging unit 13, the analysis result by the analysis unit 15, and the like. This display unit 17 is not necessarily included in the image analysis apparatus according to the present technology, and, for example, an external monitor or the like can also be used.

### (8) Imaging Object O

Although the image analysis apparatus according to the present technology may use various objects as the imaging objects, the image analysis apparatus can be suitably used for imaging an object containing, for example, a fluid as the imaging object. Due to the nature of the speckle, the speckle is not easily generated from the fluid. For this reason, when the object containing a fluid is imaged by using the image analysis apparatus 1 according to the present technology, a boundary between the fluid and other portions, a flow speed of the fluid, and the like can be obtained.

More specifically, a biological sample may be exemplified as the imaging object O, and blood may be exemplified as the fluid. For example, when the imaging apparatus 1 according to the present technology is mounted on a surgical microscope, a surgical endoscope, or the like, surgery can be performed while identifying the position of a blood vessel. Therefore, it is possible to carry out safer and highly accurate surgery, and thus, it is possible to contribute to further development of the medical technology.

Here, in a case where the imaging object O is assumed as an internal organ such as a pulsing/beating heart, and blood flowing in a blood vessel of the internal organ is analyzed by using speckle, there is a possibility that fluid imaging using speckle catches pulsations/beats and the like of the internal organ as well, with the result that the blood flow may be difficult to catch.

In contrast to the above, in the image analysis apparatus 1 according to the present technology, the motion of the fluid can be presented by the configuration of the synchronization unit 14 irrespective of pulsations/beats of the imaging object O.

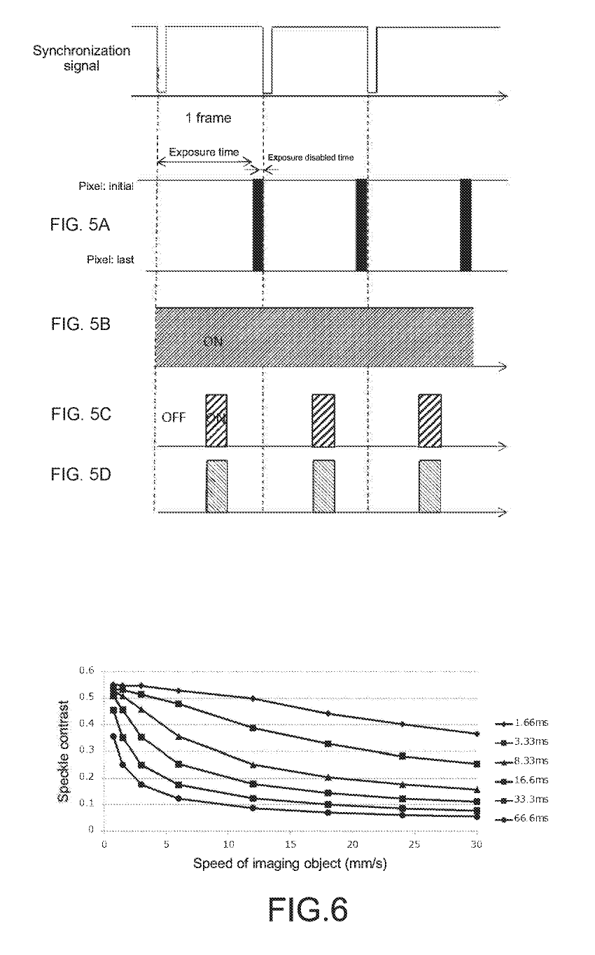

Hereinafter, an example of a drive sequence of the image analysis apparatus 1 according to the present technology that is based on the synchronization unit 14 will be described with reference to FIG. 5.

FIG. 5 is a timing chart of the image analysis apparatus 1 shown in FIG. 1, specifically, the image analysis apparatus 1 of an external modulation method in which the modulation unit 12 is provided separately from the light source 11 and in which the exposure control unit 113 is a CMOS of the global shutter system.

Note that, in FIG. 5, (a) shows an imaging time of the speckle imaging unit 13, (b) shows illumination intensity of the light source 11, (c) shows an intensity modulated time of the modulation unit 12, and (d) shows laser light illumination intensity of a modulation result.

Here, in a case where the CMOS of the global shutter system is used as the exposure control unit 113, the timing of an exposure start and the timing of an exposure end simultaneously occur in all of the pixels, and a time during which the exposure is disabled for a certain time after the end of the exposure is generated (“exposure disabled time” in FIG. 5).

For this reason, in the image analysis apparatus 1 according to the present technology, for example, the laser light is constantly emitted from the light source 11 (b), and the intensity of the laser light is modulated by using the modulation unit 12 (c). As a result, as shown in FIG. 5, the imaging object O is irradiated with the laser light whose intensity is modulated and at the same time imaging by the speckle imaging unit 13 is performed within a time during which the imaging object O can be exposed to light.

With the image analysis apparatus 1 according to the present technology configured as described above, the irradiation of the light source 11 and the imaging of the speckle imaging unit 13 are simultaneously performed by the configuration of the synchronization unit 14. For this reason, for example, even when the exposure time for the imaging object O is set to be short and the signal amount decreases, sufficient luminance can be ensured.

Further, in a case where the exposure time for the imaging object O is set to 32 ms or less, even in a situation where a pulsing/beating biological sample is used as the imaging object O and a blood flow is analyzed as a fluid, pulsations/beats of the biological sample are not caught, and only the blood flow can be caught.

More favorably, when the exposure time for the imaging object O is set to approximately 16.6 ms, while the speckle contrast decreases in motion (beat, vibration), the speckle contrast does not sufficiently decrease, and thus a decrease in contrast due to the blood flow can be caught. Still more favorably, when the exposure time for the imaging object O is set to 3.33 ms or less, the speckle contrast hardly decreases in motion (beat, vibration), and thus a decrease in speckle contrast due to the blood flow can be caught more securely.

Furthermore, in a case where the CMOS of the global shutter system is used as the exposure control unit 113, it is easy to ensure a timing at which all of the pixels are in an exposed state, and it is possible to ensure a uniform exposure amount on the entire screen.

### 2. Image Analysis Apparatus according to Second Embodiment

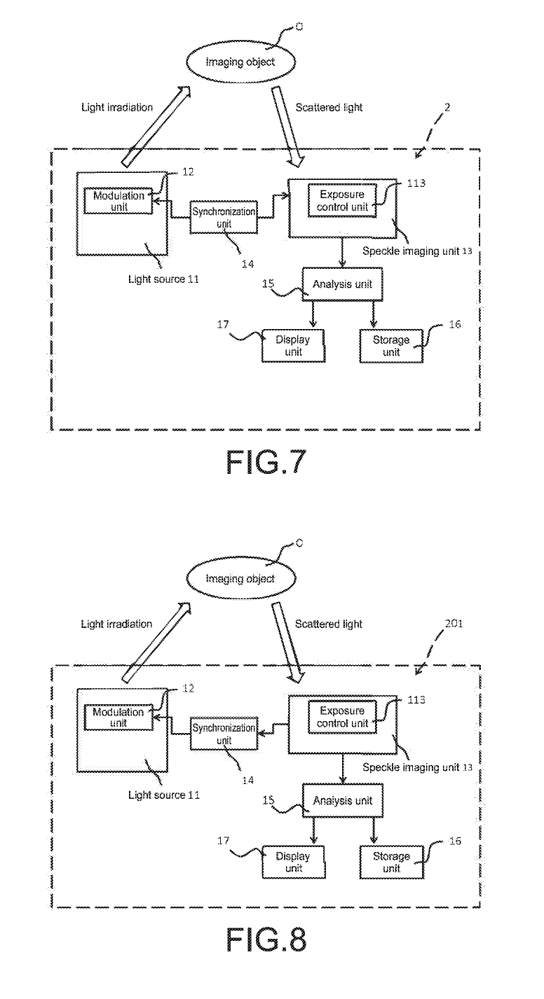

Next, a second embodiment of the image analysis apparatus according to the present technology will be described with reference to FIGS. 7 and 8. FIG. 7 is a schematic conceptual diagram schematically showing the concept of the image analysis apparatus of the second embodiment. Further, FIGS. 8 and 8 are schematic conceptual diagrams each showing a modified example of the image analysis apparatus shown in FIG. 7.

As in the image analysis apparatus 1 according to the first embodiment, the image analysis apparatus according to the second embodiment includes a light source 11, a modulation unit 12, a speckle imaging unit 13 including an exposure control unit 113, a synchronization unit 14, and an analysis unit 15. Further, the image analysis apparatus according to the second embodiment can further include a storage unit 16, a display unit 17, and the like as necessary.

Meanwhile, the image analysis apparatus according to the second embodiment is the same as the image analysis apparatuses 1, 101, 102 according to the first embodiment in the configuration of the modulation unit 12, whereas the image analysis apparatus according to the second embodiment is different from the image analysis apparatuses 1, 101, 102 according to the first embodiment in that the modulation unit 12 is incorporated in the light source 11, that is, the light source 11 is a modulation light source.

Note that, in the following description, the same configurations as those of the image analysis apparatuses 1, 101, 102 according to the first embodiment will be denoted by the same reference signs and description thereof will be omitted.

As described above, in an image analysis apparatus 2 according to the second embodiment, the light source 11 incorporates the modulation unit 12 and constitutes a so-called modulation light source. Therefore, in the image analysis apparatus 2 according to the second embodiment, laser light whose intensity is modulated is emitted from the light source 11.

Additionally, in the image analysis apparatus 2 shown in FIG. 7, a synchronization signal acquired by the synchronization unit 14 is output to the modulation unit 12 within the light source 11.

Furthermore, as in the image analysis apparatus 1 according to the first embodiment, the method of acquiring the synchronization signal is not limited. As another example of this method, a method shown in FIGS. 8 and 8 is conceivable.

### (1) Modified Example of Image Analysis Apparatus according to Second Embodiment

In other words, FIG. 8 is a schematic diagram showing a first modified example of the image analysis apparatus of the second embodiment shown in FIG. 7. As is understood from FIG. 8, this image analysis apparatus 201 employs a configuration in which the synchronization unit 14 acquires the synchronization signal from the speckle imaging unit 13.

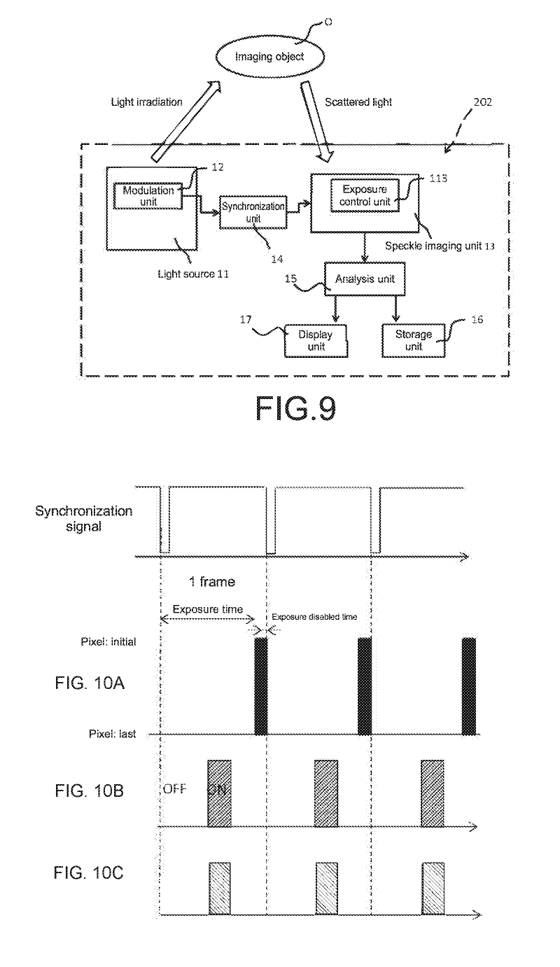

Meanwhile, FIG. 9 is a schematic diagram showing a second modified example of the image analysis apparatus of the second embodiment shown in FIG. 7. This image analysis apparatus 202 according to the second modified example employs a configuration in which the synchronization unit 14 acquires the synchronization signal from the modulation unit 12.

In those image analysis apparatuses 2, 201, 202 according to the second embodiment, the synchronization unit 14 enables an irradiation time of the light source 11 and an imaging time of the speckle imaging unit 13 to coincide with each other, and thus an analysis accuracy using speckle can be enhanced.

Hereinafter, an example of a drive sequence of the image analysis apparatus 2 according to the second embodiment will be described with reference to FIGS. 10 and 10.

FIG. 10 is a timing chart of an image analysis apparatus in which the exposure control unit 113 is a CMOS of the global shutter system, in the image analysis apparatus 2 according to the second embodiment.

Note that, in FIG. 10, (a) shows an imaging time of the speckle imaging unit 13, (b) shows illumination intensity of the light source 1, and (c) shows laser light illumination intensity of a modulation result.

As described above, since the light source 11 is a modulation light source in the image analysis apparatus 2 according to the second embodiment, laser light whose intensity is modulated is emitted. Additionally, the synchronization unit 14 causes a laser light irradiation time of the light source 11 to coincide with an imaging time of the speckle imaging unit 13.

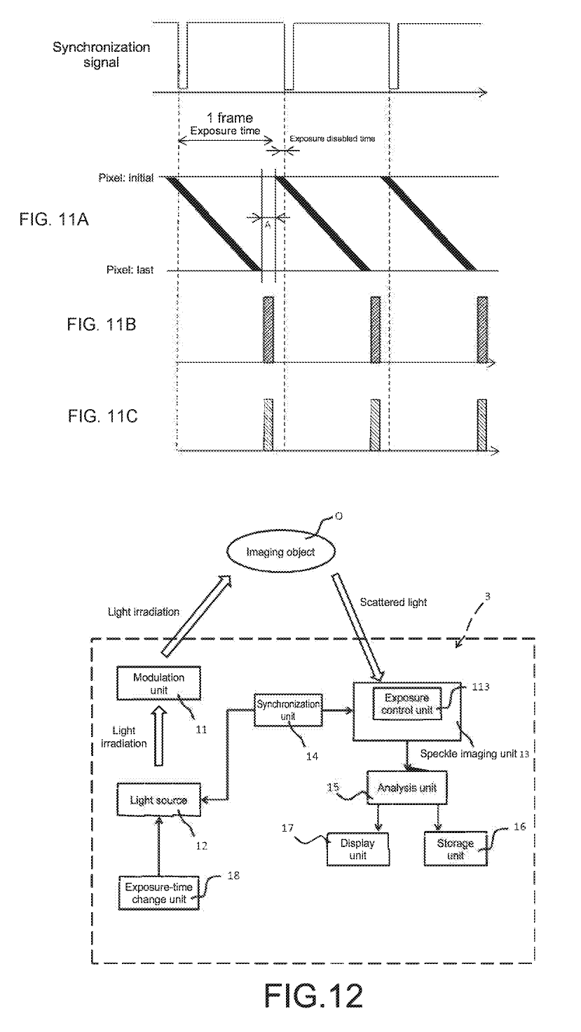

Meanwhile, FIG. 11 is a timing chart of an image analysis apparatus in which the exposure control unit 113 is a CMOS of the rolling shutter system, in the image analysis apparatus 2 according to the second embodiment.

Note that, in FIG. 11, (a) shows an imaging time of the speckle imaging unit 13, (b) shows illumination intensity of the light source 1, and (c) shows laser light illumination intensity of a modulation result.

Here, in a case where the CMOS of the rolling shutter system is used as the exposure control unit 113, the exposure start timings of the respective pixels are shifted little by little within a frame. For this reason, a time A during which all of the pixels are in the exposed state is very short (see FIG. 11).

In contrast to the above, in the image analysis apparatus 2 according to the second embodiment, the synchronization unit 14 enables the irradiation time of the light source 11 to coincide with the imaging time of the speckle imaging unit 13. Moreover, the irradiation time of the light source 11 and the imaging time of the speckle imaging unit 13 can be caused to coincide with the time A shown in FIG. 11.

With the image analysis apparatus 2 according to the present technology configured as described above, the irradiation of the light source 11 and the imaging of the speckle imaging unit 13 are simultaneously performed by the configuration of the synchronization unit 14. For this reason, for example, even when the exposure time for the imaging object 0 is set to be short and the signal amount decreases, sufficient luminance can be ensured.

Further, in a case where the exposure time for the imaging object O is set to 32 ms or less in a situation where a pulsing/beating biological sample is used as the imaging object O and a blood flow is analyzed as a fluid, the beats/pulsations of the biological sample are not caught, and only the blood flow can be caught.

Moreover, since the CMOS of the rolling shutter system is used as the exposure control unit 113, a time during which all of the pixels are in the exposed state is short. By the illumination within the short time, it is possible to catch an image with uniform illumination intensity and correctly catch blood flow information.

### 3. Image Analysis Apparatus according to Third Embodiment

Next, a third embodiment of the image analysis apparatus according to the present technology will be described with reference to FIGS. 12 and 12. As in the image analysis apparatus 1 according to the first embodiment, the image analysis apparatus 3 according to the third embodiment includes a light source 11, a modulation unit 12, a speckle imaging unit 13, a synchronization unit 14, and an analysis unit 15. Further, the image analysis apparatus 3 can further include a storage unit 16, a display unit 17, and the like as necessary.

Meanwhile, the image analysis apparatus 3 according to the third embodiment is different from the image analysis apparatuses 1, 101, 102 according to the first embodiment in that the image analysis apparatus 3 includes an exposure-time change unit 18 and in that the exposure control unit 113 is a CMOS of a rolling shutter system.

Note that, in the following description, the same configurations as those of the image analysis apparatuses 1, 101, 102 according to the first embodiment will be denoted by the same reference signs and description thereof will be omitted.

### (1) Exposure-Time Change Unit

As described above, in a case where the CMOS of the rolling shutter system is used as the exposure control unit 113, the exposure start timings of the respective pixels are shifted little by little within a frame, and thus the time A during which all of the pixels are in the exposed state is very short (see FIG. 11).

For this reason, the image analysis apparatus 3 according to the third embodiment includes an exposure-time change unit 18 that changes the exposure time for the imaging object O.

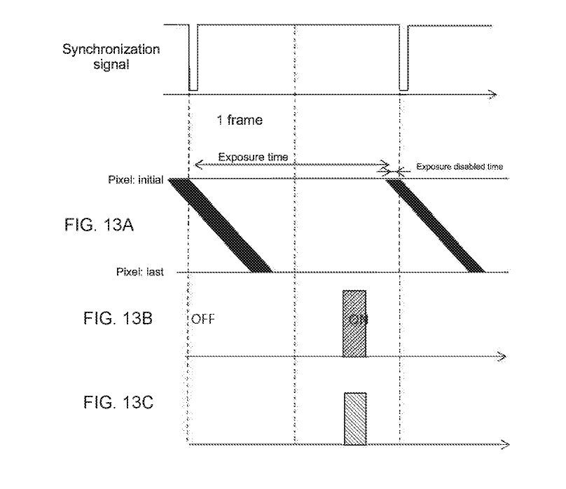

Specifically, as shown in FIG. 13, the exposure time for the imaging object O is set to be long so as to extend over two frames. When the exposure time is prolonged in such a manner, a time for exposing the imaging object O to light can be set to correspond to one frame even if the exposure control unit 113 is the CMOS of the rolling shutter system.

Note that the method of changing the exposure time by the exposure-time change unit 18 is not particularly limited, and a known method can be employed.

Additionally, as shown in FIG. 13, in the image analysis apparatus 3 according to the third embodiment, the synchronization unit 14 enables an irradiation time of the light source 11 to coincide with an imaging time of the speckle imaging unit 13. Moreover, the exposure time is prolonged by the exposure-time change unit 18, and accordingly it is easy to cause the irradiation time of the light source 11 and the imaging time of the speckle imaging unit 13 to coincide with each other.

With the image analysis apparatus 3 according to the present technology as described above, the irradiation of the light source 11 and the imaging of the speckle imaging unit 13 are simultaneously performed by the configuration of the synchronization unit 14. For this reason, for example, even when the exposure time for the imaging object O is set to be short and the signal amount decreases, sufficient luminance can be ensured.

Further, in a case where the exposure time for the imaging object O is set to 32 ms or less, even when a pulsing/beating biological sample is used as the imaging object O and a blood flow is analyzed as a fluid, the beats/pulsations of the biological sample are not caught, and only the blood flow can be caught.

Furthermore, when the exposure time for the imaging object 0 is set to approximately 16.6 ms, while the speckle contrast decreases in motion (beat, vibration), the speckle contrast does not sufficiently decrease, and thus a decrease in contrast due to the blood flow can be caught.

Besides, when the exposure time for the imaging object O is set to 3.33 ms or less, the speckle contrast hardly decreases in motion (beat, vibration), and thus a decrease in speckle contrast due to the blood flow can be caught more securely.

### 4. Image Analysis Method According to First Embodiment

The present technology also provides an image analysis method.

The image analysis method according to the first embodiment includes a modulating step, a synchronizing step, a light irradiating step, a speckle imaging step, and an analyzing step. The image analysis method may include a storing step and a displaying step as necessary. Those steps will be described in the order of actually executing the image analysis method.

### (1) Synchronizing Step

The image analysis method according to the present technology includes a synchronizing step of synchronizing an irradiation time of a light source and an imaging time of a speckle image.

Specifically, processing of inputting a synchronization signal to a light source that emits laser light being coherent light and to an imaging unit that captures a speckle image is performed.

Alternatively, a method of performing processing of acquiring a synchronization signal from the imaging unit of the speckle image and inputting the synchronization signal to the light source is exemplified. Alternatively, a method of performing processing of acquiring a synchronization signal from the light source and inputting the synchronization signal to the imaging unit is also exemplified.

### (2) Modulating Step

The image analysis method according to the present technology includes a modulating step of modulating the intensity of the laser light.

A processing method performed by this modulating step is not limited. For example, a method of changing the magnitude of a current supplied to the light source by using a semiconductor laser as a light source (direct intensity modulating method), a method of externally adding modulation to laser light that is output from a semiconductor laser being the light source (external modulation method), and the like are exemplified.

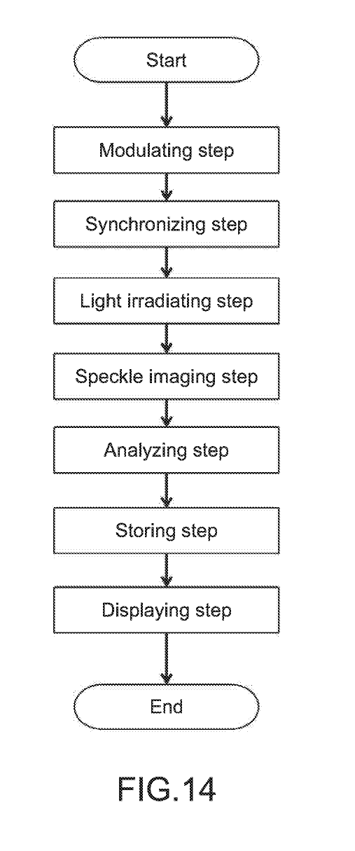

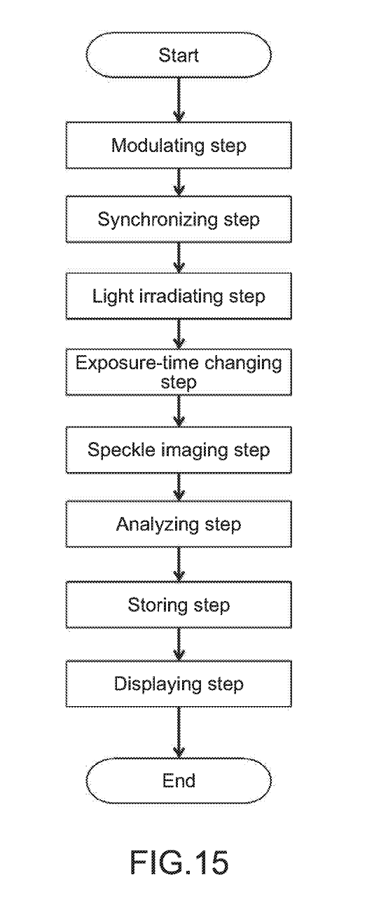

Therefore, FIG. 14 shows the direct intensity modulating method in which the modulating step is performed before the light irradiating step, but the modulating step may be allowed to be performed after the light irradiating step by the external modulation method.

### (3) Light Irradiating Step

The image analysis method according to the first embodiment includes a step of irradiating the imaging object with laser light from the light source.

Examples of the light source to be used in this light irradiating step include an argon ion (Ar) laser, a helium-neon (He—Ne) laser, a dye laser, a krypton (Cr) laser, a distributed feedback (DFB) or grating feedback semiconductor laser, and the like. Of those, it is favorable to use a semiconductor laser in which a wavelength to be output is controlled.

A modulation frequency of the light intensity in the laser light emitted in the light irradiating step is not particularly limited, but the modulation frequency needs to be adequate for speckle imaging. For example, in order to present speckle as a moving image, a modulation frequency of 24 Hz or more is favorable. In order that a user perceives a sufficiently smooth moving image, a modulation frequency of 120 Hz or more is more favorable.

Further, the exposure time for the imaging object O in the light irradiating step only needs to be set such that pulsations/beats of the imaging object shown in the background of a fluid can be suppressed and an analysis of the fluid can be performed, for example.

For example, in a case where the imaging object O is a biological sample such as a pulsing/beating heart and a state of a blood vessel of the biological sample is analyzed using speckle, it is generally understood that an arteriole has a blood flow speed of approximately 50 mm/s, whereas the pulsation/beat has approximately 1 to 5 mm/s. For this reason, in the image analysis method according to the present technology, the exposure time for the imaging object O is favorably set to 32 ms or less, more favorably, 16.6 ms or less, and further favorably, 3.33 ms or less.

### (4) Speckle Imaging Step

The image analysis method according to the first embodiment includes a speckle imaging step of capturing a speckle image on the basis of scattered light obtained by light irradiating step.

The imaging method in this speckle imaging step is not particularly limited, and one or two or more known imaging methods may be selected and used freely in combination. For example, an imaging method using an imaging element such as a CCD (Charge Coupled Device), a CMOS sensor of a global shutter system, or a CMOS sensor of a rolling shutter system may be exemplified.

In a case where a CMOS sensor of a global shutter system or a CMOS sensor of a rolling shutter system is used as the imaging element in the image analysis method according to the present technology, a state where all of the pixels in the imaging element are exposed to light can be adjusted.

In other words, in a case where the CMOS of the global shutter system is used, the timing of an exposure start and the timing of an exposure end can be caused to simultaneously occur in all of the pixels. Meanwhile, in a case where the CMOS of the rolling shutter system is used, the exposure start timings of the respective pixels are shifted little by little within a frame. For this reason, the time during which all of the pixels are in the exposed state is made short.

### (5) Analyzing Step

In the analyzing step of the image analysis method according to the first embodiment, for example, an intensity distribution of speckle is measured in a speckle image captured by the speckle imaging unit 13. Using a result of the measurement, speckle contrast, which is a value obtained by dividing the standard deviation of the intensity distribution by the average of the intensity distribution, is measured.

By such measurement of speckle contrast, angiography can be performed by using a change in speckle contrast in a case where the imaging object O is assumed as a blood vessel being a light scattering fluid. Moreover, since the speckle varies with time, the speed of the blood flow can also be analyzed.

Note that, in such a case, the method of measuring the intensity distribution of the speckle or the speckle contrast is not particularly limited as long as the effect of the present technology is not impaired, and one or two or more known measurement methods may be selected and used freely in combination.

### (6) Storing Step

The image analysis method according to the first embodiment may include a storing step as necessary.

In this storing step, the speckle image captured in the speckle imaging step, the speckle contrast measured in the analyzing step, the analysis result in the analyzing step, and the like are stored.

### (7) Displaying Step

The image analysis method according to the first embodiment may include a displaying step as necessary. In this displaying step, the speckle image captured in the speckle imaging step, the analysis result in the analyzing step, and the like are displayed on a monitor, for example.

By the image analysis method according to the first embodiment including the steps described above, since the synchronizing step is included, the irradiation of the light source and the capturing of the speckle image can be simultaneously performed. For this reason, for example, even when the exposure time for the imaging object O is set to be short and the signal amount decreases, sufficient luminance can be ensured.

Further, in a case where the exposure time for the imaging object O is set to 32 ms or less, even in a situation where a pulsing/beating biological sample is used as the imaging object O and a blood flow is to be analyzed as a fluid, the beats/pulsations of the biological sample are not caught, and only the blood flow can be caught.

Further, in a case where the CMOS of the global shutter system is used, it is easy to ensure a timing at which all of the pixels are in an exposed state, and it is possible to ensure a uniform exposure amount on the entire screen.

### 5. Image Analysis Method according to Second Embodiment

As in the image analysis method according to the first embodiment, an image analysis method according to the second embodiment may include a modulating step, a synchronizing step, a light irradiating step, a speckle imaging step, and an analyzing step and may include a storing step and a displaying step as necessary.

Meanwhile, the image analysis method according to the second embodiment is different from the image analysis method according to the first embodiment in that the image analysis method according to the second embodiment uses a CMOS of a rolling shutter system as an imaging element in the speckle imaging step and includes an exposure-time changing step of changing the exposure time for the imaging object O.

In the following description, the description of the steps common to those of the image analysis method according to the first embodiment will be omitted.

### (1) Exposure-Time Changing Step

In the image analysis method according to the second embodiment, the exposure-time changing step of changing the exposure time for the imaging object O is performed after the laser light is emitted in the light irradiating step.

Here, as described above, in a case where the CMOS of the rolling shutter system is used, the exposure start timings of the respective pixels are shifted little by little within a frame, and thus a time during which all of the pixels are in the exposed state becomes short.

Therefore, in the exposure-time changing step, for example, processing of setting the exposure time for the imaging object O to be long so as to extend over two frames is performed. When the exposure time is prolonged in such a manner, a time for exposing the imaging object O to light can be set to correspond to one frame.

By the image analysis method according to the second embodiment as described above, the irradiation of the light source and the capturing of the speckle image can be simultaneously performed by the synchronizing step.

For this reason, for example, even when the exposure time for the imaging object O is set to be short and the signal amount decreases, sufficient luminance can be ensured.

Further, in a case where the exposure time for the imaging object O is set to 32 ms or less, even in a situation where a pulsing/beating biological sample is used as the imaging object O and a blood flow is analyzed as a fluid, the beats/pulsations of the biological sample are not caught, and only the blood flow can be caught.

More favorably, when the exposure time for the imaging object O is set to approximately 16.6 ms, while the speckle contrast decreases in motion (beat, vibration), the speckle contrast does not sufficiently decrease, and thus a decrease in contrast due to the blood flow can be caught. Still more favorably, when the exposure time for the imaging object O is set to 3.33 ms or less, the speckle contrast hardly decreases in motion (beat, vibration), and thus a decrease in speckle contrast due to the blood flow can be caught more securely.

Note that the image analysis apparatus according to the present technology can also have the following configurations.
(1) An image analysis apparatus, including:

a light source that irradiates an imaging object with laser light having a controlled wavelength;

a modulation unit that modulates intensity of the laser light emitted from the light source;

a speckle imaging unit that captures a speckle image obtained from scattered light of the imaging object irradiated with the laser light;

a synchronization unit that synchronizes irradiation with the laser light and imaging by the speckle imaging unit; and an analysis unit that analyzes the speckle image captured by the speckle imaging unit.
(2) The image analysis apparatus according to (1), further including

an exposure control unit that controls an exposure time for the imaging object.
(3) The image analysis apparatus according to (1) or (2), in which

the light source irradiates the imaging object with the laser light within the exposure time for the imaging object.
(4) The image analysis apparatus according to any one of (1) to (3), in which

the exposure control unit employs a global shutter system.
(5) The image analysis apparatus according to any one of (1) to (3), in which

the exposure control unit employs a rolling shutter system.
(6) The image analysis apparatus according to any one of (1) to (5), in which

the exposure time for the imaging object is 32.2 ms or less.
(7) The image analysis apparatus according to any one of (1) to (6), in which

the light source is a distributed feedback semiconductor laser light source or a grating feedback semiconductor laser light source.
(8) An image analysis method, including:

a light irradiating step of irradiating an imaging object with laser light having a controlled wavelength;

a modulating step of modulating intensity of the laser light;

a synchronizing step of synchronizing irradiation with the laser light and imaging by a speckle imaging unit;

a speckle imaging step of capturing a speckle image obtained from scattered light of the imaging object irradiated with the laser light; and

an analyzing step of analyzing the speckle image captured by the speckle imaging unit.

### REFERENCE SIGNS LIST

1, 101, 102, 2, 201, 202, 3 image analysis apparatus
11 light source
12 modulation unit
13 speckle imaging unit
14 synchronization unit
15 analysis unit
16 storage unit
17 display unit
18 exposure-time change unit
113 exposure control unit
O imaging object

1. An image analysis apparatus, comprising:
a light source that irradiates an imaging object with laser light having a controlled wavelength;
a modulation unit that modulates intensity of the laser light;
a speckle imaging unit that captures a speckle image obtained from scattered light of the imaging object irradiated with the laser light;
a synchronization unit that synchronizes irradiation with the laser light and imaging by the speckle imaging unit; and
an analysis unit that analyzes the speckle image captured by the speckle imaging unit.

2. The image analysis apparatus according to claim 1, further comprising
an exposure control unit that controls an exposure time for the imaging object.

3. The image analysis apparatus according to claim 2, wherein
the light source irradiates the imaging object with the laser light within the exposure time for the imaging object.

4. The image analysis apparatus according to claim 3, wherein
the exposure control unit employs a global shutter system.

5. The image analysis apparatus according to claim 3, wherein
the exposure control unit employs a rolling shutter system.

6. The image analysis apparatus according to claim 4, wherein
the exposure time for the imaging object is 32.2 ms or less.

7. The image analysis apparatus according to claim 6, wherein
the light source is a distributed feedback semiconductor laser light source or a grating feedback semiconductor laser light source.

8. An image analysis method, comprising:
a light irradiating step of irradiating an imaging object with laser light having a controlled wavelength;
a modulating step of modulating intensity of the laser light;
a synchronizing step of synchronizing irradiation with the laser light and imaging by a speckle imaging unit;
a speckle imaging step of capturing a speckle image obtained from scattered light of the imaging object irradiated with the laser light; and
an analyzing step of analyzing the speckle image captured by the speckle imaging unit.
